(19) United States
(12) Patent Application Publication
(10) Pub. No.: US 2002/0094563 A1
(43) Pub. Date: Jul. 18, 2002

(54) A METHOD OF SOLID STATE FERMENTATION FOR NOMURAEA FUNGAL SPORES DEMANDING HIGH NUTRIENTS

(76) Inventors: Suey-Sheng Kao, Taipei (TW); Chien Yan Hsieh, Hsin-Ying City (TW); Yung-Sheng Tsai, Taichung Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/761,678

(22) Filed: Jan. 18, 2001

(51) Int. Cl. C12N 1/14; C12N 1/16; C12N 1/18

(52) U.S. Cl. 435/256.8; 435/254.1

(57) ABSTRACT

A solid fermentation production method for fungus spores necessitating high nutrient, including steps of mixing grain with yeast, sugar honey, fish milk and water at a certain ratio, steaming/cooking and stirring the mixture to make the grain have high nutrient content, transplanting fungus spores onto the grain after killing bacteria and solidly fermenting the fungus spores to mass-reproduce spores.

### BACKGROUND OF THE INVENTION

The present invention is related to a solid fermentation production method for fungus spores necessitating high nutrient. By means of the method, fungus spores can be mass-reproduced at low cost.

Agricultural chemicals are traditional and effective measures used as pesticides. However, such chemicals will seriously contaminate environment and ill affect human health via biological accumulation and food chain.

In order to eliminate the harm caused by traditional agricultural chemicals to human and environment, biological agents have been developed to kill pestilent insects. In such measure, the insects are infected with microbes and killed. In general, such microbes will kill only the subject insects and are harmless to other creatures.

However, there are many factors which must be considered when developing and commercializing a potential biological agent. For example, to successfully commercialize a product, there are two factors which must be considered. First, it must be possible to mass-produce the biological agent at low cost. Second, the price of the biological agent on market must be competitive. The second factor is closely related to the first factor.

It is known that nomuraea rileyi can make many kinds of pestilent insects infected and dead. According to reports, nomuraea rileyi can parasitize 30 kinds of pestilent insects, especially moths. Nomuraea rileyi can lead to infectious disease of the moths. Particularly, the half-life period of nomuraea rileyi on earth surface is up to 42 days. Therefore, it is deemed that fungus insecticide has much development potential in preservation and treatment of moths. In America, nomuraea rileyi has been used to kill larvae of moths harmful to soy bean. nomuraea rileyi is applied to wide area to induce infectious disease of moths. Such measure achieves great effect. However, nomuraea rileyi necessitates high nutrient and strict environment for production of spores. The spores can be well produced only on Sabouraud's maltose agar +1% yeast extract (SMAY ) culture medium, while on general potato dextrose agar (PDA ), nomuraea rileyi can hardly grow. Therefore, in the past, nomuraea rileyi can be only produced in factory with SMAY culture medium and no measure is available for mass-reproducing spores of nomuraea rileyi.

### SUMMARY OF THE INVENTION

It is therefore a primary object of the present invention to provide a solid fermentation production method for fungus spores necessitating high nutrient. According to this method, grain is mixed with yeast, sugar honey, fish milk and water at a certain ratio and processed to provide high nutrient content necessary for the transplanted fungus spores. Accordingly, the transplanting fungus spores can be solidly fermented to mass-reproduce spores.

The present invention can be best understood through the following description and accompanying drawings wherein:

### BRIEF DESCRIPTION OF THE DRAWINGS

FIG. 1 is a flow chart of the method of the present invention;

FIG. 2 is a photograph of the rice pack with different prescription of substrate of the present invention;

FIG. 3 is a photograph showing that the pack of the present invention is placed in a culture box for reproducing spores;

FIG. 4 is a photograph showing that nomuraea rileyi is reproduced on the rice of the present invention in one state; and

FIG. 5 is a photograph showing that nomuraea rileyi is reproduced on the rice of the present invention in another state.

### DETAILED DESCRIPTION OF THE PREFERRED EMBODIMENTS

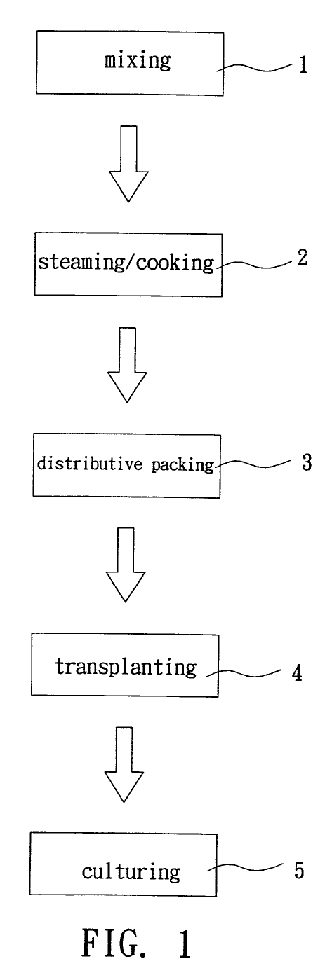

Please refer to FIG. 1 which is a flow chart of the present invention. The solid fermentation production method for fungus spores necessitating high nutrient of the present invention includes steps of:

First step 1 of mixing:

Grains are mixed with 37% ˜55% water, 4% ˜5.5% yeast, 2.5% ˜4.5% sugar honey and 0.6% ˜1.5% fish milk. In this embodiment, the grains are rice.

Second step 2 of steaming/cooking:

The mixture of rice in step 1 is steamed/cooked and stirred to a certain extent after cooled. At this time, the rice will absorb the nutrient added thereto. The rice grains are spread without adhesion on the surface. (The water content of the rice grains is about 37% ˜55%.)

Third step 3 of distributive packing:

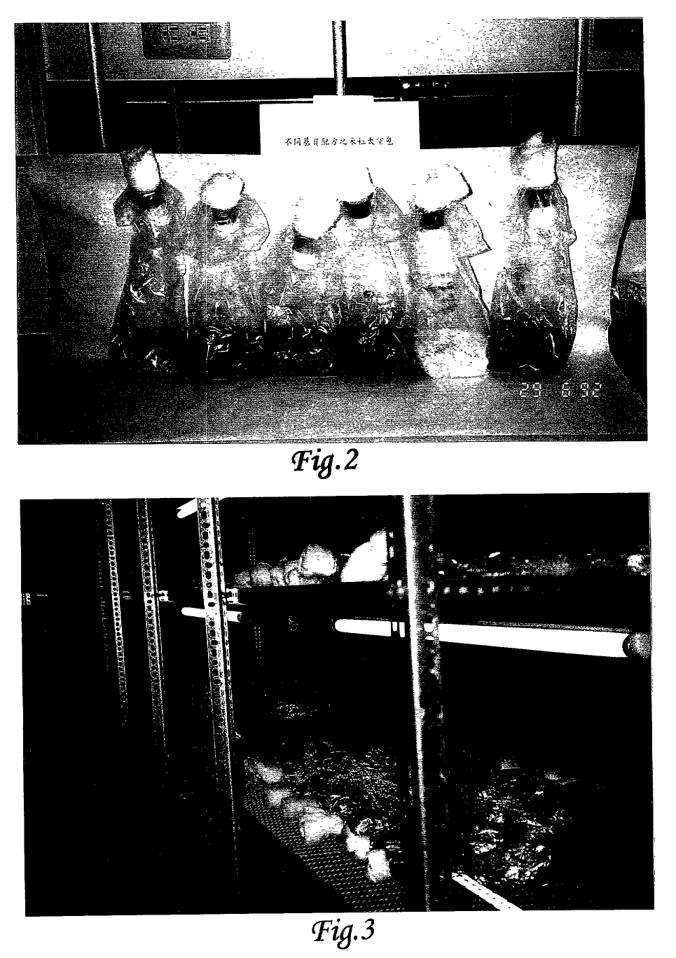

The steamed/cooked rice grains are distributively packed in wood powder packs 100˜300 grams/per pack as shown in FIG. 2. Bacteria are killed at 15 psig high pressure and 121° C. high temperature. Thereafter, the packs are cooled and ready for use.

Fourth step 4 of transplanting:

Activated fungus spores such as nomuraea rileyi are transplanted onto the rice grains in each pack. The number of the nomuraea rileyi spores transplanted in each pack is about 1˜2×10¹⁰.

Fifth step 5 of culture:

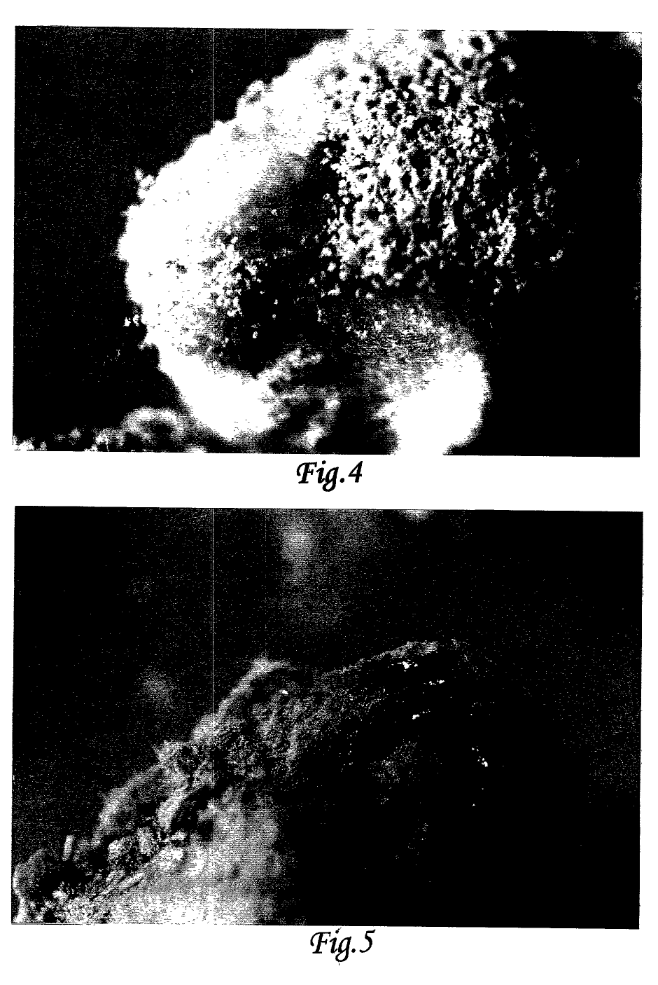

The transplanted pack is placed in a culture box as shown in FIG. 3. The culture box is kept under a condition of temperature of 24° C., relative humidity RH 70±10% and sunned 12 hours/per day. The culture is continued for 14 days to solidly ferment the spores for reproducing spores as shown in FIGS. 4 and 5.

For example, the method of the present invention is applied to the production of spores of three kinds of nomuraea rileyi TACTRIF031 (CCRC 35513 ), TACTRIF055 (CCRC 35515 ) and TACTRIFOO9 (CCRC 35509 ) necessitating high nutrient as follows:

1. With rice as the main grain for culturing nomuraea rileyi TACTRI F031 (CCRC 35513 ): After nomuraea rileyi F031 is cultured on slope face, according to the above steps, 4.5% yeast, 2.5% sugar honey and 1% fish milk are added into the rice substrate. After steamed/cooked, the water content of the rice grain is 50% and the spores are produced at 24° C. After spore stems and spores are reproduced, the color is changed from white to green and the sprouting ratio of the spores is up to 90% ˜94%. In enzyme activity test, acid phosphatase has stronger action. After production of spores is over, with this prescription, in the pack, 2.6×10¹⁰ sporeare reproduced per dry weight gram of rice grain. The spores obtained by the present invention are compared with the spores produced on SMAY culture medium via biological examination and it is found that in field test, the activity of the spores of the present invention is not apparently different from that of the comparison spores.

2. With rice as the main grain for culturing nomuraea rileyi TACTRI F055 (CCRC 35515 ): After nomuraea rileyi F055 is cultured on slope face, according to the above steps, 4% yeast, 3.5% sugar honey and 0.8% fish milk are added into the rice substrate. After steamed/cooked, the water content of the rice grain is 45% and the spores are produced at 24° C. After spore stems and spores are reproduced, the color is changed from white to green and the sprouting ratio of the spores is up to 88% ˜92%. In enzyme activity test, aphthol-AS-B1-phosphohydrolase has stronger action. After production of spores is over, with this prescription, in the pack, 3.48×10⁹ spores are reproduced per dry weight gram of rice grain. The spores obtained by the present invention are compared with the spores produced on SMAY culture medium via biological examination and it is found that in field test, the activity of the spores of the present invention is not apparently different from that of the comparison spores.

3. With rice as the main grain for culturing nomuraea rileyi TACTRI F009 (CCRC 35509 ): After nomuraea rileyi F009 is cultured on slope face, according to the above steps, 4.5% yeast, 3.0% sugar honey and 1.0% fish milk are added into the rice substrate. After steamed/cooked, the water content of the rice grain is 50% and the spores are produced at 24° C. After spore stems and spores are reproduced, the color is changed from white to green and the sprouting ratio of the spores is up to 88% ˜92%. In enzyme activity test, α-glucosidase has stronger action. After production of spores is over, with this prescription, in the pack, 8.86×10⁹ spores are reproduced per dry weight gram of rice grain. The spores obtained by the present invention are compared with the spores produced on SMAY culture medium via biological examination and it is found that in field test, the activity of the spores of the present invention is not apparently different from that of the comparison spores.

With respect to the culture of the three kinds of nomuraea rileyi TACTRIF031 (CCRC 35513 ), TACTRIF055 (CCRC 35515 ) and TACTRIF009 (CCRC 35509 ) necessitating high nutrient according to the method of the present invention, the spore productions are respectively 2.6×10¹⁰ spores/g-dry weight of rice grain, 3.48×10⁹ spores/g-dry weight of rice grain and 8.86×10⁹ spores/g-dry weight of rice grain. In comparison with 1˜2×10¹⁰ nomuraea rileyi spores transplanted in each pack of 100˜300 g, the method of the present invention apparently can reproduce a great amount of nomuraea rileyi spores.

In the present invention, rice to which nutrients are added is used as the substrate so that the cost is very low. Accordingly, the produced fungus spores have low price and high competitive ability on market.

The above embodiment is only used to illustrate the present invention, not intended to limit the scope thereof. Many modifications of the above embodiment can be made without departing from the spirit of the present invention. For example, the steamed/cooked rice can be placed in a solid fermentation tank and solidly fermented to reproduce spores.

What is claimed is:

1. A solid fermentation production method for fungus spores necessitating high nutrient, comprising steps of adding sugar honey, fish milk, yeast and water into grain, steaming/cooking the mixture, transplanting fungus spores onto the grain and culturing the fungus under a certain temperature, humidity and light control to reproduce spores.

2. A solid fermentation production method for fungus spores necessitating high nutrient, comprising steps of:
first step of mixing: mixing grain with a certain amount of water, yeast, sugar honey and fish milk;
second step of steaming/cooking: steaming/cooking the mixture of rice in first step and stirring the mixture to a certain extent after cooled;
third step of distributive packing: distributively packing the steamed/cooked grain into wood powder packs with bacteria killed at high pressure and high temperature, the packs being then cooled and ready for use;
fourth step of transplanting: transplanting activated fungus spores into each pack; and
fifth step of culture: placing the transplanted pack into a culture box and cultured for 14 days to solidly ferment the spores- for reproducing spores.

3. A solid fermentation production method for fungus spores necessitating high nutrient as claimed in claim 2, wherein in the first step, the grain is mixed with 37%˜55% water, 4%˜5.5% yeast, 2.5%˜4.5% sugar honey and 0.6%˜1.5% fish milk.

4. A solid fermentation production method for fungus spores necessitating high nutrient as claimed in claim 2, wherein in the third step, the steamed/cooked grain is distributively packed in the packs 100˜300 grams/per pack.

5. A solid fermentation production method for fungus spores necessitating high nutrient as claimed in claim 2, wherein in the third step, bacteria are killed at 15 psig high pressure and 121° C. high temperature.

6. A solid fermentation production method for fungus spores necessitating high nutrient as claimed in claim 2, wherein in the fifth step, the culture box is kept under a condition of temperature of 24° C., relative humidity RH 70±10% and sunned 12 hours/per day.

7. A solid fermentation production method for fungus spores necessitating high nutrient as claimed in claim 1, wherein the steamed/cooked grain is placed in a solid fermentation tank and solidly fermented to reproduce spores.
